(19) United States
(12) Patent Application Publication
(10) Pub. No.: US 2025/0014758 A1
(43) Pub. Date: Jan. 9, 2025

(54) METHOD AND SYSTEM FOR DETERMINING A GUT MICROBIOME MATURATION STATE OF AN INFANT

(71) Applicant: N.V. Nutricia, Zoetermeer (NL)

(72) Inventors: Francisco Manuel Codoñer Cortes, Singapore (SG); Johanna Maria Lambert, Utrecht (NL); Guus Roeselers, Utrecht (NL)

(73) Assignee: N.V. Nutricia, Zoetermeer (NL)

(21) Appl. No.: 18/823,745

(22) Filed: Sep. 4, 2024

Related U.S. Application Data
(63) Continuation of application No. PCT/EP2022/056409, filed on Mar. 11, 2022.

(51) Int. Cl. G16H 50/30; G16B 25/10

(52) U.S. Cl. CPC G16H 50/30; G16B 25/10

(57) ABSTRACT

The invention provides a method, system, and computer program product for determining a maturation state of an infant aged between 0 and 120 months, comprising

measuring at least one microbial biomarker from a set of measurable microbial biomarkers from a faecal sample of the infant,

determining a predicted age of the infant based on the at least one microbial biomarker,

wherein

the at least one microbial biomarker is selected from the set of measurable microbial biomarkers based on an analysis of the set of measurable microbial biomarkers as measured in faecal samples from a plurality of vaginally-born breast-fed infants at a plurality of ages,

the predicted age is obtained based on an evaluation of the at least one measured microbial biomarker using a function based on a plurality of corresponding at least one biomarkers measured in the faecal samples from the plurality of vaginally-born breast-fed infants at the plurality of ages.

### FIELD OF THE INVENTION

The invention relates to a method, system, and computer program product for determining a maturation state of an infant. In particular it relates to a method and system for determining a gut microbiome maturation state of an infant based on microbial biomarkers in a faecal sample, wherein the infant is between 0 and 120 months old.

### BACKGROUND OF THE INVENTION

Not all infants develop at the same rate in the early years. Therefore, two infants with the same actual age may be at different stages in their development, and may e.g. have different nutritional needs because of that. In this application, this difference is called the gut microbiome maturation state of the infant.

Moreover, if there is a significant delay between the development of an infant compared to other infants of the same actual age, this may for example be indicative of an underlying medical condition that should be diagnosed and treated as soon as possible.

Therefore, there is a need to be able to determine the gut microbiome maturation state of an infant.

### SUMMARY OF THE INVENTION

It is an object of the invention to provide a method and a system to determine a gut microbiome maturation state of an infant.

According to an aspect of the present disclosure, the invention provides a method of determining a maturation state of an infant aged between 0 and 120 months, comprising measuring at least one microbial biomarker from a set of measurable microbial biomarkers from a faecal sample of the infant, determining a predicted age of the infant based on the at least one microbial biomarker, wherein the at least one microbial biomarker is selected from the set of measurable microbial biomarkers based on an analysis of the set of measurable microbial biomarkers as measured in faecal samples from a plurality of vaginally-born breast-fed infants at a plurality of ages, the predicted age is obtained based on an evaluation of the at least one measured microbial biomarker using a function based on a plurality of corresponding at least one biomarkers measured in the faecal samples from the plurality of vaginally-born breast-fed infants at the plurality of ages.

Instead of or in addition to a maturation state, the invention may provide an estimated microbial age. The invention may provide a difference between an estimated microbial age and an actual age of the infant.

In an embodiment, the method is adapted for determining a microbial age of an infant between 0 and 120 months, or between 0 and 96 months, or between 0 and 72 months, or between 0 and 60 months, or between 0 and 48 months, or between 0 and 36 months. The method may be adapted to determine a microbial age with a granularity of 1 week, 2 weeks, 3 weeks, 4 weeks, 1 month, 2 months or 3 months. The method may be adapted to determine a reliability estimate of the determined microbial age.

Where this disclosure mentions microbial biomarkers, it is understood that they may comprise measurements concerning any biological molecules and processes. In addition, biomarkers may comprise measurements such as stool consistency, stool colour or stool pH.

In an embodiment, the analysis is performed using linear regression.

In an embodiment, the function is based on a model, preferably a machine learning model that was trained using the set of measurable microbial biomarkers as measured in faecal samples from the plurality of vaginally-born breast-fed infants at the plurality of ages.

In an embodiment, the at least one biomarker is selected based on a model, preferably a machine learning model, using the set of measurable microbial biomarkers as measured in faecal samples from the plurality of vaginally-born breast-fed infants at the plurality of ages.

In an embodiment, the, at least three microbial biomarkers from the faecal sample are used. In an embodiment, at least four, five, six, eight or ten biomarkers are used. Generally, the more microbial biomarkers used, the more accurate the model becomes (up to a certain number of microbial biomarkers).

In an embodiment, the at least one microbial biomarker is selected based on a stabilization parameter. In an embodiment, the at least one microbial biomarker is selected to minimize a the difference between the actual age and the estimated microbial age for the reference biomarker set, such as the set of vaginally-born breast-fed infants at the plurality of ages.

According to an aspect of the disclosure, the invention provides a system for determining a maturation state of an infant aged between 0 and 120 months, the system comprising:
measurement means adapted to measure at least one microbial biomarker from a set of measurable microbial biomarkers from a faecal sample of the infant,
processing means for determining a predicted age of the infant based on the at least one microbial biomarker, wherein the at least one microbial biomarker is selected from the set of measurable microbial biomarkers based on an analysis of the set of measurable microbial biomarkers as measured in faecal samples from a plurality of vaginally-born breast-fed infants at a plurality of ages, the predicted age is obtained based on an evaluation of the at least one measured microbial biomarker using a function based on a plurality of corresponding at least one biomarkers measured in the faecal samples from the plurality of vaginally-born breast-fed infants at the plurality of ages.

In an embodiment, the analysis is performed using linear regression.

In an embodiment, the function is based on a model, preferably a machine learning model that was trained using the set of measurable microbial biomarkers as measured in faecal samples from the plurality of vaginally-born breast-fed infants at the plurality of ages.

In an embodiment, the at least one biomarker is selected based on a model, preferably a machine learning model, using the set of measurable microbial biomarkers as measured in faecal samples from the plurality of vaginally-born breast-fed infants at the plurality of ages.

In an embodiment, the at least one microbial biomarker from the faecal sample comprise at least three microbial biomarkers.

In an embodiment, the at least one microbial biomarker is selected based on a stabilization parameter.

According to a further aspect of the disclosure, the invention provides a computer program product comprising machine readable instructions which, when executed on a processing device, cause said device to function according to the above described method or system.

According to a further aspect, a processing and storage system is provided, wherein the storage part comprises the reference biomarker data for determining the selected biomarkers and the microbial age evaluation function. The processing part may comprise means for accessing the data and/or for performing a statistical analysis, as described elsewhere in this disclosure, on said data. The processing part may comprise the model (e.g. the deep learning model) or other analysis instructions, as described elsewhere in this disclosure, in particular in reference to FIG. 2, that is used to determine the selected biomarkers and/or the microbial age evaluation function.

### BRIEF DESCRIPTION OF THE FIGURES

Embodiments of the present invention will be described hereinafter, by way of example only, with reference to the accompanying drawings which are schematic in nature and therefore not necessarily drawn to scale. Furthermore, like reference signs in the drawings relate to like elements.

In the figures:

FIG. 1 schematically shows depicts a system of determining the maturation state of an infant;

FIG. 2 schematically depicts a method for obtaining reference microbial biomarkers and for selecting biomarkers and a microbial age determining function, according to an embodiment of the invention;

FIG. 3 schematically depicts the method of determining the maturation state of an infant, according to an embodiment of the invention, and

FIG. 4 schematically depicts components in an electronic device that can be used to embody the invention.

### DETAILED DESCRIPTION

The human microbiome is the aggregate of the human microbiota and their genes, residing on or within human tissues and biofluids along with the corresponding anatomical sites in which they reside, including the skin and organs. The presence of certain microbial taxa and their genes may indicate the state of the particular part of the human body which said microbiome resides.

Also, the development of the microbiome is important, as it gives an indication of the development of that part of the human body for a given age. If the development is less than expected given the actual age of the human, the nutrition (e.g. through food and drink) may be adjusted to match the developmental stage of the microbiome. If the developmental stage is deviating given the actual age, then the nutrition could also be adjusted accordingly.

As the development of microbiome during infancy has an effect on the human body over its lifetime, it is recommended to determine the microbial age as early as possible, preferably already at infancy, especially if it deviates from the actual age of the child. For example, certain nutritional products aimed at children of a particular age may or may not be appropriate depending on the microbial age.

The preferred method to assess the microbial age of the baby (or infant) is to determine a gut microbiome maturation state of said baby. The gut microbiome maturation state of the baby is the predicted microbial age, obtained from the baby, versus the actual age.

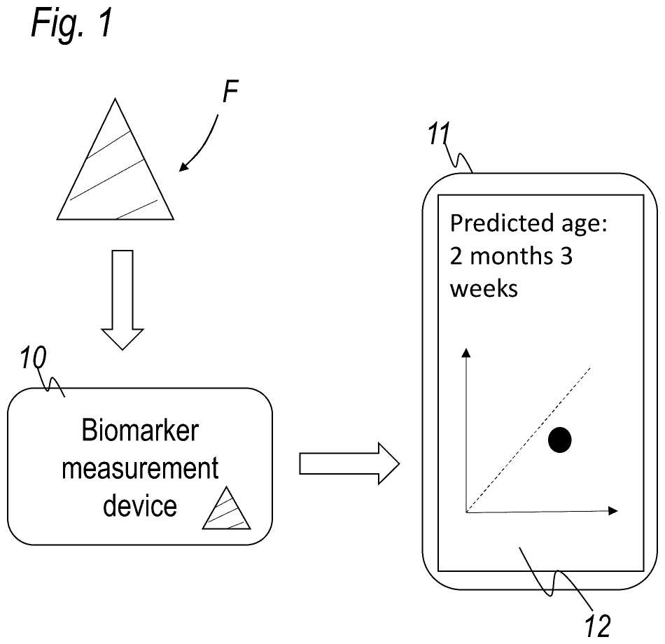

As shown in FIG. 1, this can be done by analysing biomarkers from faecal matter (F) using a biomarker measurement device 10. Multiple measurement devices (not shown) could be used to measure a variety of biomarkers. The biomarker data is then sent to an evaluation device 11 with a display 12 for displaying the result of the biomarker data evaluation. The display can comprise displaying the predicted (microbial) age, based on the evaluation of the biomarker data.

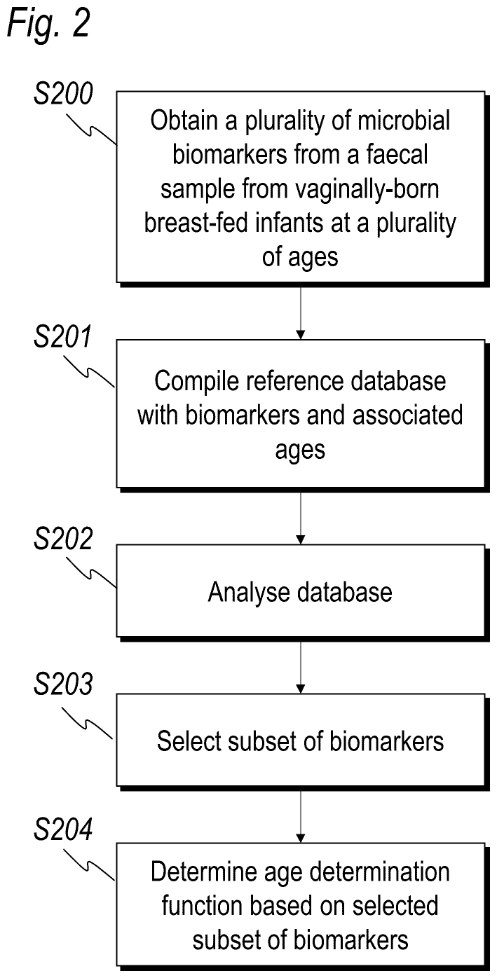

The displaying the result can comprise displaying the microbial age and the actual age of the infant, preferably in a manner that shows any difference between the two. For example, by plotting the actual age of the infant on the X axis, the determined microbial age on the Y axis, and with a diagonal line X=Y indicating a microbial age matching the actual age, any data point below the diagonal line (as in the FIG. 1) would indicate an infant with a somewhat delayed microbial development (compared to the reference) while a data point above the diagonal would indicate an infant with a somewhat advanced microbial development, again compared to a reference (which will be discussed in the context of FIG. 2).

The evaluation device 11 can be a mobile phone running a biomarker measurement and/or analysis application. In an embodiment, the biomarker measurement device 10 is integrated with the evaluation device 11, so that only a single device is needed.

In the following, it is understood that “microbial biomarkers”, unless otherwise stated, can refer to the full set of microbial biomarkers or a selected subset of the full set of microbial biomarkers which has been determined, e.g. using statistical or machine learning analysis, to be indicative of the actual age of the infant in the reference set of microbial biomarker data from vaginally-born breast fed infants.

### Obtaining Reference Microbial Biomarkers, Selecting Biomarkers, Obtaining an Age Determining Function

The method for obtaining reference microbial biomarkers, analysing and selecting biomarkers, and determining an age determination function is shown schematically in FIG. 2.

In step S200, measurements are done on faecal matter or other matter from infants with certain reference characteristics. Microbial biomarkers are obtained from the reference infants through faecal matter discharged from said baby. The faecal matter is then processed to obtain a set of reference biomarkers. While obtaining microbial biomarker data from faecal matter is preferred, it could also be obtained from other excretions and/or secretions, such as saliva, mucus, tears, skin or body surface excretions. By analysing these excretions and secretions, it is possible to determine the microbial age of the infant in a non-invasive manner.

In an embodiment, the reference biomarkers are a set of measurable microbial

biomarkers as measured in faecal samples from a plurality of vaginally-born breast-fed infants at a plurality of ages. The actual ages of the reference infants are known, and the reference infants, by definition, are considered to have a microbial age that matches their actual age. In step S201, a reference database may be compiled of the measured biomarkers and the actual ages of the reference infants. It is noted that other reference infant characteristics may be used, e.g. bottle-fed infants and C-section born infants. What is important is that the reference biomarkers are measured in samples from a group with a well-defined characteristic.

By compiling the plurality of microbial biomarkers based on the age of the (reference) infant, it is possible to generate data for all infant ages (within a specific age range) which are comparable with each other. In particular, the nutritional needs of vaginally-born, breast-fed infants over their development is well-known. By relating an actual age to a microbial age of an average vaginally-born, breast-fed infant, the nutritional needs of a child can be estimated better than from the actual age of the child alone.

In step S202, this set of reference biomarkers is subsequently analysed to determine a subset of biomarkers, possibly just one, two or three biomarkers, which are deemed most suitable for predicting the microbial and actual age of the reference infants. For example, a biomarker that remains more or less constant over the first 120 months of an infant's life is age-insensitive and therefore not suitable to predict the microbial of an infant. In contrast, a biomarker that shows a strong age dependency may be used to predict the microbial age of an infant.

Reference microbial biomarker models (hereafter “reference models” or “models”) are created by obtaining microbial biomarker information from the faecal samples of vaginally-born, breast-fed infants sampled at different stages of infant development within e.g. the initial 10 year period from birth. The temporal sampling step is preferred in units of one day, however, the step could also be that of one week, or one month, two months, three months, or any other constant or non-constant interval size.

It is preferred that the reference models contain data from infants at ages representing the initial stages of an infant, preferably the at least first 10 years after birth, more preferably at least the first (so from zero to) 8, 6, 5, 4 or 3 years.

In an embodiment, linear regression is used as part of the analysis. In another or the same embodiment, a model is used as part of the analysis, in particular a machine learning model. For example, all available measured biomarkers may be provided to a machine learning model that is then trained to determine the actual age. As part of the training, the machine learning model may be forced to select the most suitable biomarkers, so that the model learns which selected biomarkers in the full set of biomarkers are the most relevant for the age determination.

The machine learning model may be a deep learning network. The machine learning model may be an artificial neural network (ANN). It may comprise a convolutional neural network (CNN) and/or a Residual Neural Network (ResNet). The machine learning model may employ an autoencoder, with a latent space restriction which is constrained to force the model to encode only the most important subset of biomarkers. Combinations of the above may be used to form the model. In an embodiment, the trained model is analysed to determine which biomarkers are the most important. For example, in a linear or non-linear model the trained weights of the individual biomarker inputs are analysed to determine which biomarkers are most influential for determining the microbial age in the trained model.

The analysis by the model may also be performed incrementally, as new reference data is still coming in. This also allows the user to be alerted if no reference models could be generated from the reference microbial biomarker samples for some age ranges. The user then may take action to fill in (e.g. by obtaining more data samples or even through extrapolation, among other means) the missing age ranges.

The age intervals can be for each reference age (e.g. between 0 and 3, 4, 5, 6, 8 or 10 years, with a granularity of 1 day, 1 week, 1 month, 2 months, 3 months). The granularity may be finer at younger ages (e.g. 1 week for 0-3 months year old infants) that at higher ages (e.g. 1 month for above 1 year old infants).

To determine the predicted age of a new infant (an infant not included in the

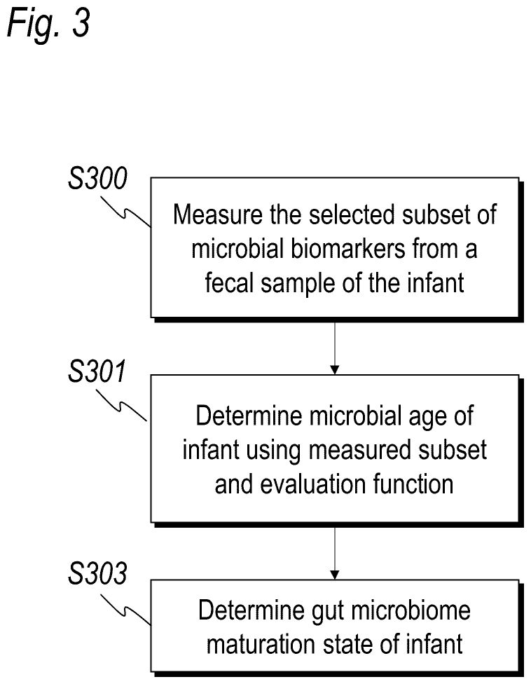

database set), would then be a matter of finding a match (using a certain metric, e.g. one generated by a machine learning model or according a statistical method) between the set of microbial biomarkers obtained in step S300 of FIG. 3 and the microbial biomarker reference database aggregate at the respective reference age. The skilled person will have other statistical tools available to accomplish the goal of determining a predicted age in step S301 based on the obtained microbial biomarkers in step S300 and the data in the reference database.

As a method to determine the robustness of the reference models, a cross-validation check of the models can be performed. Cross-validation is a resampling method that uses different portions of the data to test and train a model on different iterations. This allows the cross validation to combine (or average) to derive a more accurate estimate of model prediction performance.

As part of the analysis, a trade-off can be made regarding the number of selected biomarkers. Selecting fewer markers makes applying the method of determining a microbial age of an infant (as described in reference to FIGS. 1 and 3) easier, at the cost of some precision. Selecting more markers will generally make the result of the determination more precise, but it is in practice more elaborate to measure an increased number of biomarkers.

After the analysis of the biomarkers obtained from the reference infants is done, and regardless of whether a (human) statistical analysis, a linear or non-linear regression analysis, or a machine learning model is employed, the following is determined: 1) as part of step S203, a subset of one or more biomarkers among the full set of measurable biomarkers which is deemed sufficient for determining a microbial age at a desired reliability and 2) as part of step S204, an evaluation function (model, mathematical function, chart, etc) for determining an infant's microbial age based on measurements of the selected biomarkers from the infant. It is noted that step S203 may also fully or partially take place after step S204. Step S203 may comprise a manual (hand-picked) (pre) selection and/or a machine (e.g. by a model) selection of biomarkers. This improves the flexibility in selecting the most optimal subset to perform the necessary analysis.

In an embodiment, the subset of microbial biomarkers is selected based on one or more stabilization parameters. For example, the subset may be based on one or more hand-picked biomarkers which are known to predict a microbial age. Such biomarkers could then, for example, be assigned more weight when predicting the microbial age.

The evaluation function (hereafter: “the function”) for determining the infant's microbial age will generally be in a digital form, e.g. as the weights in a trained linear or non-linear model, as a programmed mathematical function, etc. The digital form of the means can be embodied in many ways, e.g. as part of the evaluation device (e.g.

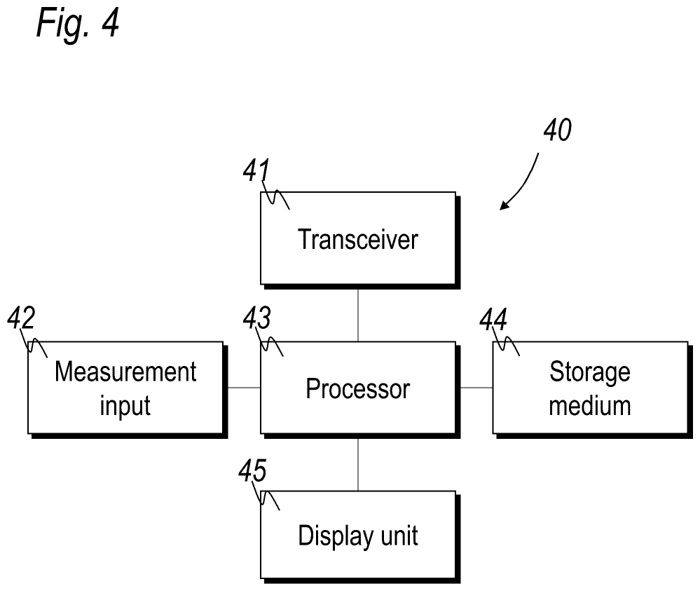

mobile phone) 11 of FIG. 1 or the device that will be used in FIG. 3 or 4 (see below), as a separate server that is accessed by said portable device over a network, or in any other digital processing means available to the skilled person. The function may be a procedure implemented in programming code to be run on a processor.

In an embodiment, at different ages different biomarkers or determination

functions are selected. For example, it may be that on average for infants between the ages of 0 and 12 months different biomarkers are optimal for microbial age prediction than for infants of 12 months and older.

In an embodiment, the analysis of the reference microbial biomarkers results in a reference profile of selected biomarkers for a set of ages (e.g. a range of ages at 1 month or 1 week granularity). The function for determining the microbial age could then involve a search for the most relevant match between the biomarkers measured in an infant with unknown microbial age and the set of reference profiles with associated reference ages, or it could be a general (trained) function taking the selected biomarkers as input.

### Determining the Gut Microbiome Maturation State of an Infant

The steps for determining the predicted microbial age and the gut microbiome maturation state of the infant are shown schematically in FIG. 3. The predicted (microbial) age is the age determined by analysing the selected microbial biomarkers present in the faecal matter.

First, in step S300 the selected subset of biomarkers (e.g. one or more biomarkers deemed most relevant, based on the reference database, for determining a microbial age of an infant) is measured. This may be done using a measurement device, e.g. such as the device 10 shown in FIG. 1. The measurement device is configured to perform the task of identifying microbial biomarkers from faecal matter. Any device may be able to perform this, provided that the device comprises a means to receive faecal matter, identify the microbial biomarkers, and generate a data file suitable for use by the electronic device. For example, the faecal matter could be analysed by a dedicated, stand-alone machine, or could even be transported to a lab for analysis, with the lab sending the data file the electronic device.

The output in this step may be a data file comprising all of the microbial biomarkers that are present in the faecal matter of the baby. Alternatively, it may comprise only the selected biomarkers, as determined in the analysis of step S202 and S203. In other words, it may be determined that only a subset of microbial biomarkers may be necessary for determining a reliable microbial age, and may therefore generate a data file based on a partial selection of all the microbial biomarkers from the original data file. This generation of a new data file may be performed on the portable mobile device, a personal computer (PC), or through cloud computing or any other electronic and/or virtual device.

Examples of microbial biomarkers are bifidobacteriaceae spp., butyrate and lactic acid-producing bacteria.

In step S301, the determined function is used to determine the microbial age of the infant based on the measured subset of biomarkers. This may be done in the same device as step S301, or in a different evaluation device, e.g. a portable mobile device 11 as shown in FIG. 1. If a different device is used, the data file with the measured biomarkers can be provided over a wired or wireless network, or on a portable physical storage medium for analysis on the portable electronic device. The output of the analysis could be a display screen outputting the predicted age and a visualised result of the maturation state, in the form of a graphical output.

According to an embodiment of the present invention, in step S301 the predicted microbial age is determined based on the plurality of microbial biomarkers available in the faecal sample F. This determination can use any of the functions that have been described in more detail in the context of FIG. 2.

While it is generally preferred to use a pre-trained model as the microbial age determination function, it is also possible to do this training on the fly, starting from the reference database of FIG. 2. Moreover, some of the options discussed in reference to FIG. 2 do not use a pre-trained model. For example, if as part of the analysis the reference database is organised in reference biomarker profiles for a set of reference ages, then the microbial age determination may comprise finding the reference age associated with the reference biomarker profile that most closely matches (using a suitable matching metric function to calculate a distance between biomarker sets) the measured subset of biomarkers. This is also a convenient method in case some biomarkers are not always available. A matching metric function can be designed which accounts for missing biomarker data in the measured subset.

Furthermore, the input of an actual age may also narrow the range of possible ages in the determination step of S301, leading to a more accurate prediction of the age. For example, as was discussed in the context of FIG. 2, when at different ages, different biomarkers or determination functions are selected, a coarse indication of the infant's age can be used as input to make sure that the right biomarker subset and/or age determination function is used.

By comparing the actual age (for example requested by the analysing device as user input) of the infant with the determined microbial age, in step S302 the gut microbiome maturation state of the infant is determined. The result may be output onto a display screen of the electronic device. The output may also include the deviation of the predicted age with the actual age (i.e. the gut microbiome maturation state) in the form of a graph, so the information may be more easily understood by the user.

Preferably, three or more microbial biomarkers are used in the selected subset of microbial biomarkers.

### Applications of the Gut Microbiome Maturation State of an Infant

Once the maturation state of an infant is determined, the system may be configured to provide recommendations for increasing/decreasing of the abundance of certain microbial biomarkers for a particular infant. This could be performed by suggesting certain diets, or supplements. In an embodiments, these recommendations are automated in e.g. the evaluation device 11, for example as part of a programmed expert system or other recommendation system.

The gut microbiome maturation state could be used to warn of a significant gap between predicted age and actual age. Such a gap could be indicative of an underlying medical condition that is responsible for a “microbial age” (predicted age) to be significantly lower (or, higher) than the actual age.

According to another aspect of the present invention, a system for determining the gut microbiome maturation state of the infant is described. The system may be a computer implementation of the method as described in reference to FIGS. 1, 2 and/or 3.

FIG. 4 depicts a portable electronic device 40 according to an embodiment of the present invention. The system can embody devices 10 and/or 11 of FIG. 1. The portable electronic device may comprise a transceiver 41 for receiving the microbial biomarker data and for transmitting/receiving information which may be used when determining the predicted age. The portable electronic device may have an optional measurement input 42 for analysing faecal matter or for obtaining the result of the faecal analysis done by another device (not shown in FIG. 4). The portable electronic device further comprises a storage medium 44 for storing the microbial biomarker data and other data such as data related to the evaluation function, and a processor 43 configured to perform the steps of predicting the age of the infant, based on the microbial biomarker data in the received faecal sample (microbial biomarker) data, and to determine the maturation state of the infant. The processor may also be configured to process the machine learning algorithms described herein. The portable electronic device may also comprise a display unit 45, configured to output the maturation state and/or the predicted age of the infant.

The displayed output may be in the form of text (alphanumeric) as well as visual output, such as graphs and charts. As shown in FIG. 1, the maturation state may be displayed on a graph, representing the predicted age against actual age. The display unit may also be configured with an interactive module such that the user may select information on the display to perform additional processes. For example, once the predicted age is displayed, the display unit may output the microbial biomarkers used for the calculation. The user may then select a subset of the microbial biomarkers, or other microbial biomarkers not used in the prediction, and prompt the portable electronic device to perform the prediction again (using the processor).

In the foregoing description of the figures, the invention has been described with reference to specific embodiments thereof. It will, however, be evident that various modifications and changes may be made thereto without departing from the scope of the invention as summarized in the attached claims.

In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

In particular, combinations of specific features of various aspects of the invention may be made. An aspect of the invention may be further advantageously enhanced by adding a feature that was described in relation to another aspect of the invention.

It is to be understood that the invention is limited by the annexed claims and its technical equivalents only. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the element is present, unless the context clearly requires that there be one and only one of the elements. The indefinite article “a” or “an” thus usually means “at least one”.

1. A method for determining a maturation state of an infant aged between 0 and 120 months, comprising
measuring at least one microbial biomarker from a set of measurable microbial biomarkers from a faecal sample of the infant,
determining a predicted age of the infant based on the at least one microbial biomarker, wherein
the at least one microbial biomarker is selected from the set of measurable microbial biomarkers based on an analysis of the set of measurable microbial biomarkers as measured in faecal samples from a plurality of vaginally-born breast-fed infants at a plurality of ages,
the predicted age is obtained based on an evaluation of the at least one measured microbial biomarker using a function based on a plurality of corresponding at least one biomarkers measured in the faecal samples from the plurality of vaginally-born breast-fed infants at the plurality of ages.

2. The method of claim 1, wherein the analysis is performed using linear regression.

3. The method of claim 1, wherein the function is based on a model, preferably a machine learning model that was trained using the set of measurable microbial biomarkers as measured in faecal samples from the plurality of vaginally-born breast-fed infants at the plurality of ages.

4. The method of claim 3, wherein the at least one biomarker is selected based on the model.

5. The method of any claim 1, wherein at least three microbial biomarkers from the faecal sample are used.

6. The method of any claim 1, wherein the at least one microbial biomarker is selected based on a stabilization parameter. 7: (Original) A system for determining a maturation state of an infant aged between 0 and 120 months, the system comprising:
measurement means adapted to measure at least one microbial biomarker from a set of measurable microbial biomarkers from a faecal sample of the infant,
processing means for determining a predicted age of the infant based on the at least one microbial biomarker, wherein
the at least one microbial biomarker is selected from the set of measurable microbial biomarkers based on an analysis of the set of measurable microbial biomarkers as measured in faecal samples from a plurality of vaginally-born breast-fed infants at a plurality of ages,
the predicted age is obtained based on an evaluation of the at least one measured microbial biomarker using a function based on plurality of corresponding at least one biomarkers measured in the faecal samples from the plurality of vaginally-born breast-fed infants at the plurality of ages.

8. The system of claim 7, wherein the analysis is performed using linear regression.

9. The system of claim 7, wherein the function is based on a model, preferably a machine learning model that was trained using the set of measurable microbial biomarkers as measured in faecal samples from the plurality of vaginally-born breast-fed infants at the plurality of ages.

10. The system of claim 9, wherein the at least one biomarker is selected based on the model.

11. The system of any claim 7, wherein at least three microbial biomarkers from the faecal sample are used.

12. The system of any claim 7, wherein the at least one microbial biomarker is selected based on a stabilization parameter.

13. A computer program product comprising machine readable instructions which, when executed on a processing device, cause said device to execute the following functions;
measuring at least one microbial biomarker from a set of measurable microbial biomarkers from a faecal sample of the infant.
determining a predicted age of the infant based on the at least one microbial biomarker, wherein
the at least one microbial biomarker is selected from the set of measurable microbial biomarkers based on analysis of set of measurable microbial biomarkers as measured in faecal samples from a plurality of vaginally-born breast-fed infants at a plurality of ages,
the predicted age is obtained based on an evaluation of the at least one measured microbial biomarker using a function based on a plurality of corresponding at least one biomarkers measured in the faecal sample from the plurality of vaginally-born breast-fed infants at the pluarality of ages.

14. The computer program of claim 13, wherein the analysis is performed using linear regression.

15. The computer program of claim 13, wherein the function is based on a model, preferably a machine learning model that was trained using the set of measurable microbial biomarkers as measured in faecal samples from the plurality of vaginally-born breast-fed infants at the plurality of ages.

16. The computer program of claim 15, wherein the at least one biomarker is selected based on the model.

17. The computer program of claim 13, wherein at least three microbial biomarkers from the faecal sample are used.

18. The computer program of claim 13, wherein the at least one microbial biomarker is selected based on a stabilization parameter.
